(19) United States
(12) Patent Application Publication
(10) Pub. No.: US 2024/0215875 A1
(43) Pub. Date: Jul. 4, 2024

(54) WEARABLE DEVICE AND A METHOD FOR SELECTING AND INTERPRETING LIGHT INTENSITY DATA VALUES APPLICABLE THERETO

(71) Applicants: NATIONAL TAIPEI UNIVERSITY OF TECHNOLOGY, TAIPEI CITY (TW); TAIPEI MEDICAL UNIVERSITY, TAIPEI CITY (TW)

(72) Inventors: CHENG-CHUN CHANG, TAIPEI CITY (TW); PO-WEN LU, TAIPEI CITY (TW)

(21) Appl. No.: 18/400,268

(22) Filed: Dec. 29, 2023

(30) Foreign Application Priority Data
Dec. 30, 2022 (TW) 111150853

(51) Int. Cl. A61B 5/1455; A61B 5/00

(52) U.S. Cl. CPC A61B 5/1455; A61B 5/681; A61B 5/7203; A61B 5/7267; A61B 2560/0443; A61B 2562/0238; A61B 2562/0242

(57) ABSTRACT

A wearable device and a method for selecting and interpreting light intensity data values applicable thereto. The wearable device includes a light emitting unit and a spectrum sensing unit. The method includes steps of: controlling the light-emitting unit to simultaneously emit a mixed light including multiple spectrum frequency bands to enter inside skin of the user; controlling the spectrum sensing unit to sense the intensity of an outgoing light from inside skin of the user at a series of sampling time to generate a spectrum data set including a plurality of groups of frequency band-light intensity data values; and selecting at least one of a first group of frequency band-light intensity data values satisfying a signal quality index in the spectrum data set to perform a data interpretation at a first judgment time point.

### BACKGROUND OF THE INVENTION

### 1. Field of the Invention

The present application relates to a wearable device and a method for selecting and interpreting light intensity data values applicable thereto, and especially to a wearable device applicable to a user' body and a method for selecting and interpreting light intensity data values applicable thereto.

### 2. The Prior Arts

With increasing popularity of wearables devices, many people wear smart watches or smart bracelets. Most of these wearable devices already have functions of monitoring physiological values for measuring and recording various data that can represent changes in the user's physical status, thereby monitoring the user's health status in real time.

Please refer to FIG. 1, which is a functional block diagram illustrating a conventional wearable device contacting the user's skin, wherein a bottom of the wearable device 11 contacts a surface of the user's skin 10. A photoplethysmography (hereafter PPG) module is usually set on the bottom of a main body 11 of the wearable device. In the conventional technical means, a traditional PPG module 110 only emits a light of single frequency band into inside skin at single time point, then detects an intensity change of outgoing light from the inside skin, thereby estimates a status of blood vessels in dermis and subcutaneous tissue, and finally estimates physiological status such as heart rate, pulse or blood pressure.

For example, when a light beam of certain wavelength irradiates on the skin surface, vasoconstriction and vasodilatation caused by each heartbeat will affect transmission of the light (such as to perform transmissive PPG to the light passing through fingertips) or reflection of the light (such as to perform reflective PPG to the light reflected from near wrist surface). When the light irradiates the skin tissue then is reflected to a light sensor, the intensity of the light will attenuate to a certain extent. When there is no large-scale movement of the measuring site, the light absorption of muscles, bones, veins and other connective tissue is basically unchanged, but arteries are different, there is obvious blood flow in arteries, the light absorption of that has significant change naturally. When the light is converted to electrical signals through PPG method, the signals obtained can be divided into DC signals and AC signals, precisely because the light absorption of arteries changes and that of the other tissues basically remain. Therefore, as long as the AC signals are extracted, characteristics of the blood flow can be reflected.

However, the light sensor for receiving the outgoing light in the traditional PPG module 110 can only convert the received outgoing light of single frequency band into a single signal curve in energy intensity change. Moreover, a displacement that is easily occurred between the wearable device and the skin causes change of an incident distance, so the measuring result is easily distorted due to the user's shaking. Furthermore, because sensing information that can be generated by the outgoing light of the single frequency band is not rich enough, the aforesaid deficiencies will eventually lead to misjudgments in the wearable device, which will make it impossible to monitor the user's health status in real time and effectively.

### SUMMARY OF THE INVENTION

How to solve the aforesaid deficiencies in the traditional photoplethysmography module is the main purpose of developing technical means of the present application. The present invention main relates to a wearable device, which includes: a main body, including a casing, which includes a sensing surface that can be close to outside skin of the user; a physiological data sensing module, disposed in the casing, including a light emitting unit and a spectrum sensing unit, wherein the light emitting unit is used to simultaneously emit a mixed light including multiple spectrum frequency bands, and the mixed light enters inside skin of the user through the sensing surface; the spectrum sensing unit is used to sense an intensity of an outgoing light from inside skin of the user at a series of sampling time to generate a spectrum data set, the spectrum data set includes a plurality of light intensity data values, the plurality of light intensity data values can be divided into a plurality of groups of frequency band-light intensity data values respectively obtained corresponding to the plurality of spectrum frequency bands, and can be respectively divided into a plurality of sampling time-light intensity data value sets corresponding to the series of sampling time; and an arithmetic unit, with signal connecting to the physiological data sensing module, for receiving the spectrum data set; according to a signal quality of the plurality of groups of frequency band-light intensity data in the spectrum data set, the arithmetic unit selects at least one of a first group of frequency band-light intensity data values satisfying a signal quality index in the spectrum data set to perform a data interpretation at a first judgment time point, and selects at least one of a second group of frequency band-light intensity data values satisfying the signal quality index in the spectrum data set to perform the data interpretation at a second judgment time point.

According to the above concept, in the wearable device recited in the present application, the sensing surface is against outside wrist skin of the user, the physiological data sensing module is a micro spectrometer, the light emitting unit in the micro spectrometer includes light emitting diodes that is used to simultaneously emit the mixed light including multiple spectrum frequency bands, the spectrum sensing unit in the micro spectrometer is used to sense the outgoing light from inside skin of the user at the series of sampling time to generate a plurality of spectrum frequency bands-intensity signal curves, any one of the spectrum frequency bands-intensity signal curves is generated from one group of the frequency band-light intensity data values in the spectrum data set.

According to the above concept, in the wearable device recited in the present application, the arithmetic unit is disposed in the casing, signal connecting to the physiological data sensing module, for receiving the spectrum data set; according to a signal quality of the plurality of spectrum frequency band-intensity signal curves generated from the spectrum data set, the arithmetic unit selects one or more of the spectrum frequency band-intensity signal curves of the signal quality meeting a condition in the spectrum data set to perform the data interpretation at the first judgment time point, and additionally selects one or more of frequency band-intensity signal curves of the signal quality meeting the condition in the spectrum data set to perform the data interpretation at the second judgment time point; the signal quality the plurality of spectrum frequency band-intensity signal curves is its signal-to-noise ratio value.

According to the above concept, in the wearable device recited in the present application, in any one of the plurality of spectrum frequency band-intensity signal curves, signal components within a frequency band range from a lower limit frequency to an upper limit frequency are defined as the signal, and signal components outside the frequency band range from the lower limit frequency to the upper limit frequency are defined as the noise; the arithmetic unit respectively calculates a ratio of signal intensity and noise intensity of each the spectrum frequency band-intensity signal curve of the plurality of spectrum frequency band-intensity signal curves to obtain the signal-to-noise ratio value, then selects and determines one or more of the spectrum frequency band-intensity signal curves of the signal-to-noise ratio value greater than a preset value as a qualified spectrum frequency band-intensity signal curve, and performs a PPG data interpretation to one or more of the qualified spectrum frequency band-intensity signal curves.

According to the above concept, in the wearable device recited in the present application, the lower limit frequency is 0.5 Hz, the upper limit frequency limit is 7 Hz, the arithmetic unit performs a maximum ratio combining of the plurality of frequency band-intensity signal curves which meet a signal-to-noise ratio standard for weighting and combining them into an optimized intensity signal curve, and then uses the optimized intensity signal curve to perform the PPG data interpretation subsequently.

According to the above concept, in the wearable device recited in the present application, the PPG data interpretation can estimate one value of white blood cell count, red blood cell count, hemoglobin, hematocrit, blood glucose, blood pressure, blood urea nitrogen, creatinine and alanine aminotransferase (ALT) of the user.

According to the above concept, in the wearable device recited in the present application, the PPG data interpretation is optimized by using artificial intelligence learning, and simultaneously generates p spectrum frequency band-intensity signal curves corresponding p spectrum frequency bands to provide more data in a short time for training of an artificial intelligence program, which can quickly improve accuracy of the data interpretation.

According to the above concept, in the wearable device recited in the present application, the arithmetic unit determines whether measuring environment or device wearing conditions are correct according to a sampling time-light intensity data value sets corresponding to the plurality of spectrum frequency bands obtained at the same sampling time point.

According to the above concept, in the wearable device recited in the present application, the arithmetic unit determines measuring object characteristics according to a sampling time-light intensity data value sets corresponding to the plurality of spectrum frequency bands obtained at the same sampling time point, and selects an appropriate artificial intelligence model and prediction model according to the measuring object characteristics.

According to the above concept, in the wearable device recited in the present application, the arithmetic unit determines measuring object characteristics according to a sampling time-light intensity data value sets corresponding to the plurality of spectrum frequency bands obtained at the sampling time point, and selects a measuring parameter according to the measuring object characteristics, the measuring parameter is a light source intensity of an individual frequency band, a sampling speed and measuring times of the spectrum sensing unit, a lens aperture value or exposure value.

According to the above concept, in the wearable device recited in the present application, the arithmetic unit converts the spectrum data set into a flat color image, two-dimensional coordinates of the flat color image are the sampling time and the spectrum frequency band respectively, a color value of any coordinate point in the flat color image is a light intensity value, and the arithmetic unit perform the data interpretation to the flat color image by using a image recognition technology of convolutional neural network.

Another aspect of the present application is a wearable device, applicable to a user, including: a main body, including a casing, which includes a sensing surface that can be close to outside skin of the user; a physiological data sensing module, disposed in the casing, including a light emitting unit and a spectrum sensing unit, wherein the light emitting unit is used to emit an incident light of intensity changing with time, the incident light of different intensities enters inside skin of the user to different depths through the sensing surface; the spectrum sensing unit is used to sense an intensity of an outgoing light from inside skin of the user at a series of sampling time to generate a spectrum data set, the spectrum data set includes a plurality of light intensity data values, the plurality of light intensity data values can be divided into a plurality of groups of depth-light intensity data values respectively obtained corresponding to the plurality of incident light intensities; and an arithmetic unit, with signal connecting to the physiological data sensing module, for receiving the spectrum data set; according to a signal quality of the plurality of groups of depth-light intensity data in the spectrum data set, the arithmetic unit selects at least one of a first group of depth-light intensity data values satisfying a signal quality index in the spectrum data set to perform a data interpretation at a first judgment time point, and selects at least one of a second group of depth-light intensity data values satisfying the signal quality index in the spectrum data set to perform the data interpretation at a second judgment time point.

A further aspect of the present application is a method for selecting and interpreting light intensity data values, applicable between a user and a wearable device, the wearable device includes a light emitting unit and a spectrum sensing unit, the method includes following steps of: controlling the light emitting unit simultaneously emit a mixed light including multiple spectrum frequency bands, and the mixed light enter inside skin of the user through a sensing surface; controlling the spectrum sensing unit sense an intensity of an outgoing light from inside skin of the user at a series of sampling time to generate a spectrum data set, wherein the spectrum data set includes a plurality of light intensity data values, the plurality of light intensity data values can be divided into a plurality of groups of frequency band-light intensity data values respectively obtained corresponding to the plurality of spectrum frequency bands, and can be respectively divided into a plurality of sampling time-light intensity data value sets corresponding to the series of sampling time; and according to a signal quality of the plurality of groups of frequency band-light intensity data in the spectrum data set, selecting at least one of a first group of frequency band-light intensity data values satisfying a signal quality index in the spectrum data set to perform a data interpretation at a first judgment time point, and selecting at least one of a second group of frequency band-light intensity data values satisfying the signal quality index in the spectrum data set to perform the data interpretation at a second judgment time point.

Another aspect of the present application is a method for selecting and interpreting light intensity data values, applicable between a user and a wearable device, the wearable device includes a light emitting unit and a spectrum sensing unit, the method includes following steps of: controlling the light emitting unit emit, and incident light of intensity changing with time, wherein the incident light of different intensities enters inside skin of the user to different depths through the sensing surface; controlling the spectrum sensing unit sense an intensity of an outgoing light from inside skin of the user at a series of sampling time to generate a spectrum data set, wherein the spectrum data set includes a plurality of light intensity data values, the plurality of light intensity data values can be divided into a plurality of groups of depth-light intensity data values respectively obtained corresponding to the plurality of incident light intensities; and according to a signal quality of the plurality of groups of depth-light intensity data in the spectrum data set, selecting at least one of a first group of depth-light intensity data values satisfying a signal quality index in the spectrum data set to perform a data interpretation at a first judgment time point, and selecting at least one of a second group of depth-light intensity data values satisfying the signal quality index in the spectrum data set to perform the data interpretation at a second judgment time point.

In order to have a more clear understanding of the aforesaid concepts of the present invention, embodiments are provided below and described in detail accompanying with corresponding drawings.

### BRIEF DESCRIPTION OF THE DRAWINGS

FIG. 1 is a functional block diagram illustrating a conventional wearable device contacting the user's skin.

FIG. 2a is a functional block schematic diagram illustrating a wearable device developed by the present application.

FIG. 2b is a functional block schematic diagram illustrating a light emitting unit developed by the present application.

FIG. 2c is a spectrogram of the three light sources.

FIG. 2d is a schematic diagram illustrating elements configuration of the physiological data sensing module developed by the present application.

FIG. 3a is a schematic diagram illustrating incident light emitted by the wearable device of the present application.

FIG. 3b is a diagram illustrating a representative example of outgoing light intensity distribution of three different frequency bands.

FIG. 3c is a schematic diagram illustrating three spectrum frequency band-intensity signal curves that change with time and correspond to different spectrum bands generated by the device of the present application.

FIG. 4 is a flow chart illustrating a preferred embodiment of a method for selecting and interpreting light intensity data values developed by the present application.

FIG. 5 is a flow chart illustrating another preferred embodiment of the method for selecting and interpreting light intensity data values developed by the present application.

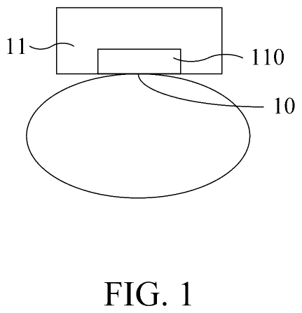

### DETAILED DESCRIPTION OF THE PREFERRED EMBODIMENT

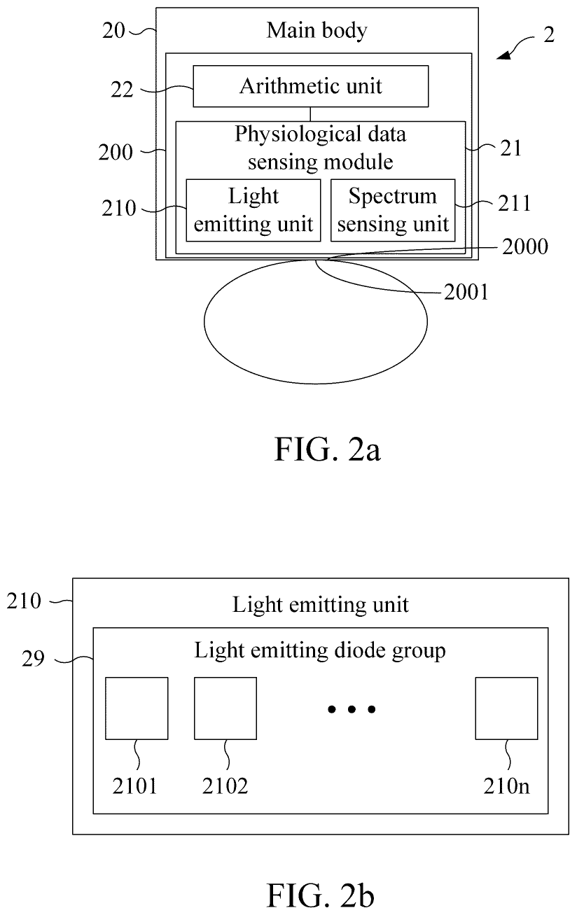

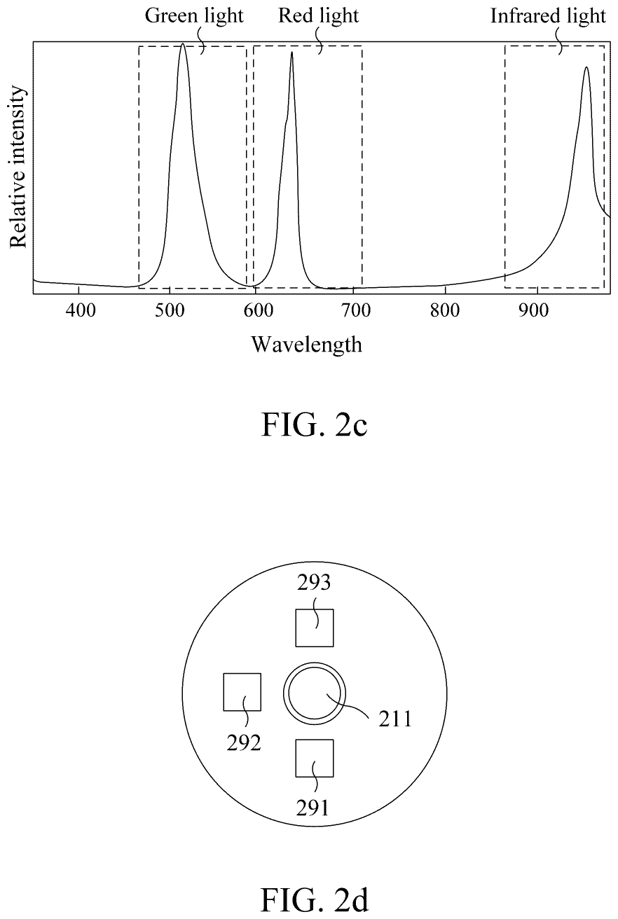

For resolve the aforesaid problems, the inventor of the present application develops a wearable device having functional blocks as shown in FIG. 2a, which can be applicable to physiological data sensing. The wearable device mainly includes a main body 20, a physiological data sensing module 21 and an arithmetic unit 22, wherein the main body 20 includes a casing 200, the casing 200 includes a sensing surface 2000, and the sensing surface 2000 can be close to or even against skin surface of the user 2001.

As for the physiological data sensing module 21, it can be disposed in the casing 200, and includes a light emitting unit 210 and a spectrum sensing unit 211, the light emitting unit 210 is mainly used to simultaneously emit a mixed light including multiple spectrum frequency bands. For example, the light emitting unit 210 can be equipped with a single light emitting diode of a frequency band capable of covering multiple-bands spectra (such as infrared, visible light and ultraviolet); or as a functional block schematic diagram illustrating a light emitting unit shown in FIG. 2b, the light emitting unit 210 can include a light emitting diode group 29 composed of plural light emitting diodes 2101, 2102 . . . 210n with different frequency bands. For example, the plural light emitting diodes can be the light emitting diodes with green light (wavelength 515 nm), red light (wavelength 660 nm) and infrared light (wavelength 940 nm) respectively, a spectrogram of the three light sources as shown in FIG. 2c.

The mixed light of multiple spectrum frequency bands simultaneously emitted by the light emitting unit 210 enters the skin surface of the user 2001 through the sensing surface 2000. Since the incident light of different frequency bands can enter inside skin of the user to different depths, an outgoing light intensity change of different frequency bands (wavelength range) included in an outgoing mixed light from inside the skin surface of the user will carry the physiological status changes representing that occur at different depths. In order to receive light intensity signals in all frequency bands, the spectrum sensing unit 211 can be a Multi-Wavelength Photoplethysmography (hereafter MW-PPG) sensor, it can be completed with, for example, a micro spectrometer developed by nanoLambda company. As shown in FIG. 2d, the light emitting diode group 29 composed of the light emitting diodes 291, 292, 293 of green light (wavelength 515 nm), red light (wavelength 660 nm) and infrared light (wavelength 940 nm) can be disposed in the physiological data sensing module 21 developed by the present application, the light sources are arranged symmetrically; and the spectrum sensing unit 211 is placed in the middle, so that the outgoing light of different wavelengths from the inside skin can be gathered at center point to facilitate collection by the MW-PPG sensor. Compared with an area of the traditional spectrometer easily being several square centimeters, an area of this sensor only accounts for a few square millimeters, its structure does not require to use precision optical components such as grating, focal lenses; it is small and lightweight in use, and it can simultaneously collects the PPG signals of 153 wavelengths including 340 nm, 345 nm . . . 505 nm, 510 nm, 515 nm, 520 nm, 525 nm, 620 nm, 625 nm, 630 nm, 635 nm, 640 nm, 930 nm, 935 nm, 940 nm, 945 nm, 950 nm.

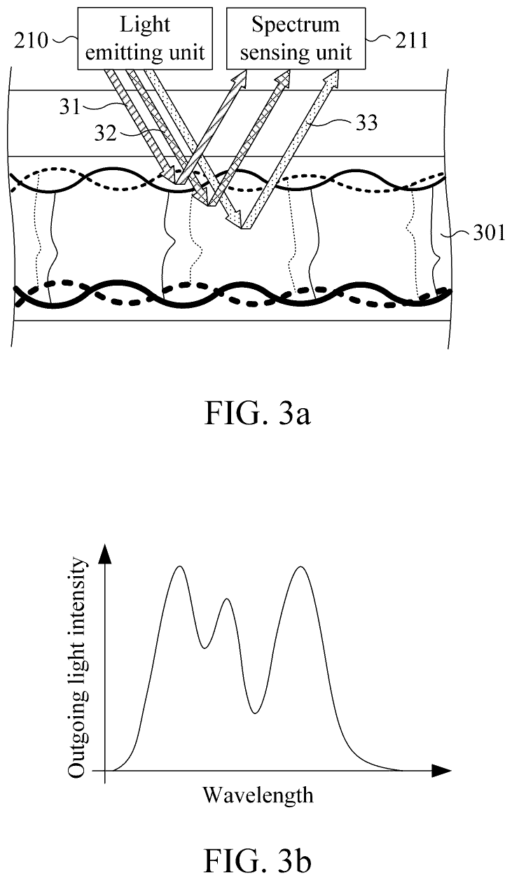

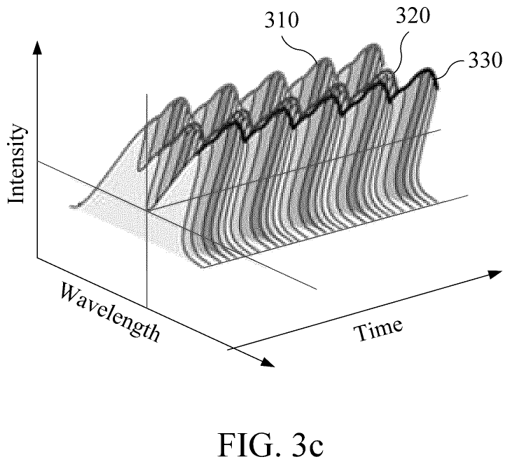

To take a schematic diagram of the incident light shown in FIG. 3a as an example, the incident light 31, 32, 33 of three different frequency bands (wavelength range) can enter different depths of dermis 301 in the user's skin, an outgoing light intensity distribution of the outgoing mixed light from inside the skin surface of the user at a certain sampling time point is as shown in FIG. 3b, in which the three peaks represent the main wavelength position of the incident light 31, 32, 33 of three different frequency bands (wavelength range). Since the incident light emitted by the light emitting unit 210 is the mixed light includes multiple frequency bands (infrared rays and visible light) at the same time, the outgoing light is also belong to the mixed light of multiple frequency bands. Therefore, the spectrum sensing unit 211 of the present application can be used to sense the outgoing mixed light of multiple-bands spectra from inside the skin surface of the user at a series of sampling time points T1, T2, . . . , Tn, and to generate a spectrum data set, the spectrum set includes n sampling time-light intensity data value sets corresponding to the series of sampling time points T1, T2, . . . , Tn.

For example, the first sampling time-light intensity data value group in the n sampling time-light intensity data value groups is composed of p light intensity data values corresponding to p spectrum frequency bands (F₁, F₂, . . . ,Fₚ) respectively obtained at the first sampling time point T₁. As for the second sampling time-light intensity data group in the n sampling time-light intensity data value groups, it is composed of p light intensity data values corresponding to p spectrum frequency bands (F₁, F₂, . . . ,Fₚ) respectively obtained at the second sampling time point T₂. According to a signal quality of the plurality of groups of frequency band-light intensity data in the spectrum data set, the arithmetic unit 22 can select at least one of the first group of frequency band-light intensity data values satisfying a signal quality index in the spectrum data set to perform data interpretation at a first judgment time point, and can also select at least one of the second group of frequency band-light intensity data values satisfying the signal quality index in the spectrum data set to perform the data interpretation at a second judgment time point. In this way, even if the relative displacement occurred between the wearable device and the user due to weak wearing causes variation in arrival depth of the incident light, at least a group of frequency band-light intensity data values measured with the best depth at that time can be instantly select to perform the data interpretation according to the signal quality, so that the misjudgments caused by depth variations can be avoided; or at least a group of frequency band-light intensity data values with the best measuring effect at that time can be instantly select to perform the data interpretation according to the signal quality, so that the misjudgments caused by measuring environmental changes, physiological changes or depth variations can be avoided.

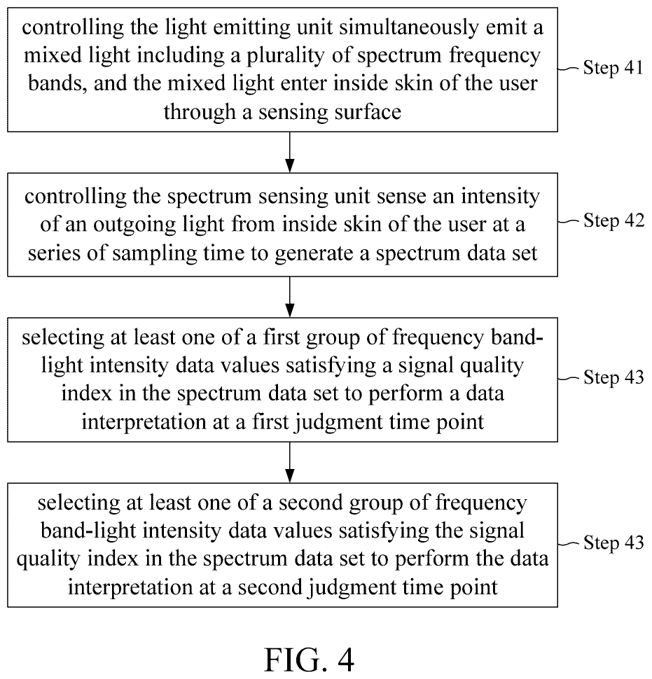

In other words, p spectrum frequency band-intensity signal curves corresponding to the p spectrum frequency bands (F₁, F₂, . . . , Fₚ) can be generated according to the spectrum data set. To take the above FIG. 3a and FIG. 3b as examples, after the series of sampling time, three spectrum frequency band-intensity signal curves 310, 320, 330 as shown in FIG. 3c that vary over the time and correspond to different spectrum frequency bands can be obtained. From the above description and FIGS. 3a-3c, it can be seen that the spectrum data set obtained by utilizing the hardware setting of the present application can generates the p spectrum frequency band-intensity signal curves representing different skin depths; in this way, the arithmetic unit 22 with signal connecting to the physiological data sensing module 21 (the arithmetic unit 22 can be disposed in the casing 200 or in a cloud) can receive the spectrum data generated by the aforesaid spectrum sensing unit 211. In this way, according to the signal quality of the plurality of p groups of frequency band-light intensity data in the spectrum data set, the arithmetic unit 22 can select at least one of the first group of frequency band-light intensity data values satisfying the signal quality index in the spectrum data set to perform the data interpretation at the first judgment time point, and can also select at least one of the second group of frequency band-light intensity data values satisfying the signal quality index in the spectrum data set to perform the data interpretation at the second judgment time point. The aforesaid method for selecting and interpreting light intensity data values can be applicable between the user and the wearable device, a flow chart of the method can refer to FIG. 4, and the method includes following steps of: controlling the light emitting unit simultaneously emit a mixed light including a plurality of spectrum frequency bands, and the mixed light enter inside skin of the user through a sensing surface (step 41); controlling the spectrum sensing unit sense an intensity of an outgoing light from inside skin of the user at a series of sampling time to generate a spectrum data set (step 42), wherein the spectrum data set includes a plurality of light intensity data values, the plurality of light intensity data values can be divided into a plurality of groups of frequency band-light intensity data values respectively obtained corresponding to the plurality of spectrum frequency bands, and can be respectively divided into a plurality of sampling time-light intensity data value sets corresponding to the series of sampling time; then, according to a signal quality of the plurality of groups of frequency band-light intensity data in the spectrum data set, selecting at least one first group of frequency band-light intensity data values satisfying a signal quality index in the spectrum data set to perform a data interpretation at a first judgment time point (step 43), and selecting at least one second group of frequency band-light intensity data values satisfying the signal quality index in the spectrum data set to perform the data interpretation at a second judgment time point (step 44).

For example, the arithmetic unit 22 can use signal-to-noise ratio (SNR) in the signal quality to select at least a spectrum frequency band-intensity signal curve from the p spectrum frequency band-intensity signal curves corresponding to the p spectrum frequency bands to perform the PPG data interpretation. As for the above application example, in any one of the spectrum frequency band-intensity signal curves, signal components within the frequency band range from a lower limit frequency to an upper limit frequency are defined as the signal, and signal components outside the frequency band range from the lower limit frequency to the upper limit frequency are defined as the noise. For example, if the spectrum frequency band-intensity signal curve represents the signals of blood vessel pulse, the signal component within the frequency range 0.5 Hz˜7 Hz can be defined as the signal, and the signal component outside the frequency range 0.5 Hz˜7 Hz can be defined as the noise, so the signal-to-noise ratio (SNR) can be respectively calculated for each one of the p spectrum frequency band-intensity signal curves, then at least one of the spectrum frequency band-intensity signal curves of the signal-to-noise ratio greater than a preset value is selected and determined as a qualified spectrum frequency band-intensity signal curve, and the data interpretation (e.g. the PPG data interpretation) is performed to one or more of the qualified spectrum frequency band-intensity signal curve. Another embodiment is to perform a maximum ratio combining (MRC) of the plurality of frequency band-intensity signal curves which meet a signal-to-noise ratio standard according to the signal-to-noise ratio for weighting and combining them into an optimized intensity signal curve, and then to use the optimized intensity signal curve to perform the data interpretation subsequently. The aforesaid PPG data interpretation to the blood vessel changes in the skin of the present application has more information to perform training and judging, so it can also be widely applicable to estimate one value of white blood cell count, red blood cell count, hemoglobin, hematocrit, blood glucose, blood pressure, blood urea nitrogen, creatinine and alanine aminotransferase (ALT) of the user.

The data interpretation of the arithmetic unit 22 performing to the qualified spectrum frequency-intensity signal curves or the optimized intensity signal curves can be the widely used PPG data interpretation, and a data interpretation which is continuously optimized by using artificial intelligence learning (AI learning) and has various cardiovascular status-related data applied thereto. An artificial neural network (ANN) is a mathematical model that imitates structures and functions of human brain and is mainly used to process problems such as regression and classification, with increasing of computing speed and performance of hardware devices such as graphic cards, number of layers of the neutral network model can be greatly deepened, so a number of neuron features that can be trained can also increase accordingly, and thus the neural network model has opportunities to obtain better learning outcomes. However, in the medical field, each information is not easily collected, and amount of the training data is an important key to determining the quality of the model. Therefore, the wearable device completed by the technical means of the present application can simultaneously generate the p spectrum frequency band-intensity signal curves corresponding to the p spectrum frequency bands, more data for training of the artificial intelligence program can be provided in a short time, and accuracy of the data interpretation will be quickly improved.

More preferably, the present application can be optimized by using a transfer learning method. The transfer learning method belongs to a special research field in machine learning, its research source is based on a point of view: when human beings encounter new problems, if they have encountered similar or identical problems in the past, they can more quickly and efficiently solve the new problems based on the experience and knowledge they have learned; that is a process of transferring knowledge. Therefore, as long as a model same as or with tasks related to the neural network model desired to be trained can be found, the model can be used as a source domain model and to train the target domain model through the transfer learning, it is no need to collect data, label data and train model for the target domain from scratch, and the time of collecting data is greatly saved. Therefore, If the amount of data collected by using the micro-spectrometer of the present application is still insufficient, and the results of direct training of the neural network model are usually still unsatisfactory, the present application can use data of MMIC open database (same as “Y. Kurylyak, F. Lamonaca and D. Grimaldi, “A neural network-based method for continuous blood pressure estimation from a PPG signal,” in proc. of 2013 IEEE I2MTC, Minneapolis, MN, pp. 280-283, 2013. ”) through the transfer learning method as the training data for a source domain neural network model.

Additionally, through observation of the intensity signal curves 310, 320, 330 that change with time and correspond to the plurality of spectrum frequency bands shown in FIG. 3c, three-dimensional PPG intensity distribution signals can be seen, if the intensity (height) is converted into a color value, the three-dimensional image can be converted into a flat color image, and the flat color image can be interpreted by using powerful AI image recognition technologies such as convolutional neural network (CNN). Similarly, after the light intensity data values in the spectrum data set were converted into color values, the spectrum data set can also be converted into a flat color image to perform the aforesaid data interpretation.

Furthermore, the arithmetic unit 22 of the present application can determines whether the measuring environment or device wearing conditions are correct according to the p light intensity data values (hereafter snapshot spectrum) corresponding to the p spectrum frequency bands (F1, F2, . . . ,Fp) obtained at a certain sampling time point, can also determine measuring object characteristics (such as race) according to the current snapshot spectrum for selecting the appropriate artificial intelligence models and prediction models. Of course, a measuring parameter can also be selected according to the measuring object characteristics (such as race) determined by the current snapshot spectrum, the various measuring parameters can be, for example, a light source intensity of individual frequency band, sampling speed and measuring times of the spectrum sensing unit 211, even adjustment of lens aperture value for control exposure value. These means are all intended to make the measuring results closer to real state and reduce the chance of misjudgment.

About examples of the signal quality, in addition to determine the signal quality with the direct and convenient signal-to-noise ratio (SNR), an empirical rule can also be used to select, the empirical rule is to compare waveforms with past waveforms and to eliminate it that is too different, or to compare the intensity signal curve of a certain frequency band with the intensity signal curve of a frequency band adjacent thereto, and to eliminate it that is too different. Furthermore, a hindsight determining method can also be used, for example, if the heart rate value, the blood oxygen value or any blood indicator currently predicted after the data interpretation using the intensity signal curve of a certain frequency band is abnormal, significantly beyond possible range value of human beings, the intensity curve of the frequency band can be eliminated, and the intensity signal curves of the remaining frequency bands can be instead used to perform the data interpretation; by repeating such process, the best frequency band signal can be iteratively selected.

In addition to use the mixed light of multiple frequency bands to irradiate the skin to different depths thereof, the light emitting unit 210 of the present application can also be controlled to emit the incident light of the same frequency but different intensities at different time periods (for example, in a time-division multiplexing mode, the incident light of different intensities are emitted in different time slots; certainly, the incident light of different frequency bands and different intensities can also be used. Then, the incident light of different intensities can enter inside skin of the user to different depths through the sensing surface 2000. The spectrum sensing unit 211 can be used to sense an intensity of an outgoing light from inside skin of the user at a series of sampling time to generate a spectrum data set, the spectrum data set includes a plurality of light intensity data values, the plurality of light intensity data values can be divided into a plurality of groups of depth-light intensity data values respectively obtained corresponding to the plurality of incident light intensities. In this way, according to a signal quality of the plurality of groups of depth-light intensity data in the spectrum data set, the arithmetic unit of the present application can select at least one first group of depth-light intensity data values satisfying a signal quality index in the spectrum data set to perform a data interpretation at a first judgment time point, and select at least one second group of depth-light intensity data values satisfying the signal quality index in the spectrum data set to perform the data interpretation at a second judgment time point. In this way, even if the relative displacement occurred between the wearable device and the user due to weak wearing causes variation in arrival depth of the incident light, at least a group of frequency band-light intensity data values measured with the best depth at that time can be instantly select to perform the data interpretation according to the signal quality, so that the misjudgments caused by depth variations can be avoided.

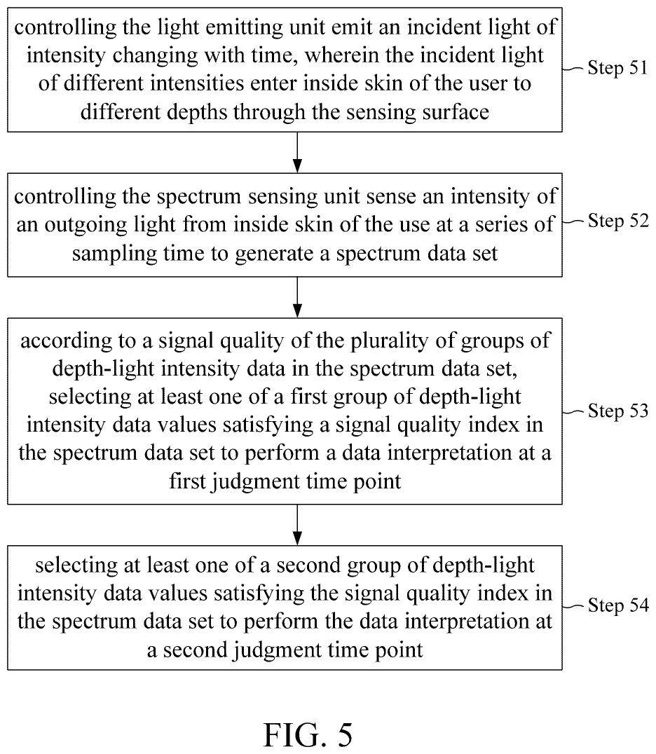

The aforesaid method for selecting and interpreting light intensity data values can be applicable between the user and the wearable device, a flow chart of the method can refer to FIG. 5, and the method includes following steps of: controlling the light emitting unit emit an incident light of intensity changing with time, wherein the incident light of different intensities enter inside skin of the user to different depths through the sensing surface (step 51); controlling the spectrum sensing unit sense an intensity of an outgoing light from the user's skin at a series of sampling time to generate a spectrum data set (step 52), wherein the spectrum data set includes a plurality of light intensity data values, the plurality of light intensity data values can be divided into a plurality of groups of depth-light intensity data values respectively obtained corresponding to the plurality of incident light intensities; then, according to a signal quality of the plurality of groups of depth-light intensity data in the spectrum data set, selecting at least one first group of depth-light intensity data values satisfying a signal quality index in the spectrum data set to perform a data interpretation at a first judgment time point (step 53), and selecting at least one second group of depth-light intensity data values satisfying the signal quality index in the spectrum data set to perform the data interpretation at a second judgment time point (step 54).

In summary, the technology of the present application can improve distortion of the measuring results of the wearable device caused by changes of the incident distance due to displacement between it and the skin, and generate a rich amount of sensing information by using the outgoing light of multiple frequency bands or light intensities to select the better signal, so as to improve the shortcoming of being prone to misjudgment of the traditional wearable device, and to achieve the effects of real-time and effective monitoring of the user' health status. Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A wearable device, applicable to a user, comprising:
a main body, comprising a casing, which comprises a sensing surface that can be close to outside skin of the user;
a physiological data sensing module, disposed in the casing, comprising a light emitting unit and a spectrum sensing unit, wherein the light emitting unit is used to simultaneously emit a mixed light comprising multiple spectrum frequency bands, and the mixed light enters inside skin of the user through the sensing surface; the spectrum sensing unit is used to sense an intensity of an outgoing light from inside skin of the user at a series of sampling time to generate a spectrum data set, the spectrum data set comprises a plurality of light intensity data values, the plurality of light intensity data values can be divided into a plurality of groups of frequency band-light intensity data values respectively obtained corresponding to the plurality of spectrum frequency bands; and
an arithmetic unit, with signal connecting to the physiological data sensing module, for receiving the spectrum data set; according to a signal quality of the plurality of groups of frequency band-light intensity data in the spectrum data set, the arithmetic unit selects at least one of a first group of frequency band-light intensity data values satisfying a signal quality index in the spectrum data set to perform a data interpretation at a first judgment time point, and selects at least one of a second group of frequency band-light intensity data values satisfying the signal quality index in the spectrum data set to perform the data interpretation at a second judgment time point.

2. The wearable device according to claim 1, wherein the sensing surface is against outside wrist skin of the user, the physiological data sensing module is a micro spectrometer, the light emitting unit in the micro spectrometer comprises light emitting diodes that is used to simultaneously emit the mixed light comprising multiple spectrum frequency bands, the spectrum sensing unit in the micro spectrometer is used to sense the outgoing light from inside skin of the user at the series of sampling time to generate a plurality of spectrum frequency bands-intensity signal curves, any one of the spectrum frequency bands-intensity signal curves is generated from one group of the frequency band-light intensity data values in the spectrum data set.

3. The wearable device according to claim 2, wherein the arithmetic unit is disposed in the casing, signal connecting to the physiological data sensing module, for receiving the spectrum data set; according to a signal quality of the plurality of spectrum frequency band-intensity signal curves generated from the spectrum data set, the arithmetic unit selects one or more of the spectrum frequency band-intensity signal curves of the signal quality meeting a condition in the spectrum data set to perform the data interpretation at the first judgment time point, and additionally selects one or more of frequency band-intensity signal curves of the signal quality meeting the condition in the spectrum data set to perform the data interpretation at the second judgment time point; the signal quality the plurality of spectrum frequency band-intensity signal curves is its signal-to-noise ratio value.

4. The wearable device according to claim 3, wherein, in any one of the plurality of spectrum frequency band-intensity signal curves, signal components within a frequency band range from a lower limit frequency to an upper limit frequency are defined as the signal, and signal components outside the frequency band range from the lower limit frequency to the upper limit frequency are defined as the noise; the arithmetic unit respectively calculates a ratio of signal intensity and noise intensity of each the spectrum frequency band-intensity signal curve of the plurality of spectrum frequency band-intensity signal curves to obtain the signal-to-noise ratio value, then selects and determines one or more of the spectrum frequency band-intensity signal curves of the signal-to-noise ratio value greater than a preset value as a qualified spectrum frequency band-intensity signal curve, and performs a PPG data interpretation to one or more of the qualified spectrum frequency band-intensity signal curves.

5. The wearable device according to claims 4, wherein the PPG data interpretation can estimate one value of white blood cell count, red blood cell count, hemoglobin, hematocrit, blood glucose, blood pressure, blood urea nitrogen, creatinine and alanine aminotransferase (ALT) of the user.

6. The wearable device according to claims 4, wherein the PPG data interpretation is optimized by using artificial intelligence learning, and simultaneously generates p spectrum frequency band-intensity signal curves corresponding p spectrum frequency bands to provide more data in a short time for training of an artificial intelligence program, which can quickly improve accuracy of the data interpretation.

7. The wearable device according to claim 4, wherein the lower limit frequency is 0.5 Hz, the upper limit frequency limit is 7 Hz, the arithmetic unit performs a maximum ratio combining of the plurality of frequency band-intensity signal curves which meet a signal-to-noise ratio standard for weighting and combining them into an optimized intensity signal curve, and then uses the optimized intensity signal curve to perform the PPG data interpretation subsequently.

8. The wearable device according to claims 7, wherein the PPG data interpretation can estimate one value of white blood cell count, red blood cell count, hemoglobin, hematocrit, blood glucose, blood pressure, blood urea nitrogen, creatinine and alanine aminotransferase (ALT) of the user.

9. The wearable device according to claims 7, wherein the PPG data interpretation is optimized by using artificial intelligence learning, and simultaneously generates p spectrum frequency band-intensity signal curves corresponding p spectrum frequency bands to provide more data in a short time for training of an artificial intelligence program, which can quickly improve accuracy of the data interpretation.

10. The wearable device according to claim 1, wherein the arithmetic unit determines whether measuring environment or device wearing conditions are correct according to a sampling time-light intensity data value set corresponding to the plurality of spectrum frequency bands obtained at the same sampling time point.

11. The wearable device according to claim 1, wherein the arithmetic unit determines measuring object characteristics according to a sampling time-light intensity data value sets corresponding to the plurality of spectrum frequency bands obtained at the same sampling time point, and selects an appropriate artificial intelligence model and prediction model according to the measuring object characteristics.

12. The wearable device according to claim 1, wherein the arithmetic unit determines measuring object characteristics according to a sampling time-light intensity data value sets corresponding to the plurality of spectrum frequency bands obtained at the sampling time point, and selects a measuring parameter according to the measuring object characteristics, the measuring parameter is a light source intensity of an individual frequency band, a sampling speed and measuring times of the spectrum sensing unit, a lens aperture value or exposure value.

13. The wearable device according to claim 1, wherein the arithmetic unit converts the spectrum data set into a flat color image, two-dimensional coordinates of the flat color image are the sampling time and the spectrum frequency band respectively, a color value of any coordinate point in the flat color image is a light intensity value, and the arithmetic unit perform the data interpretation to the flat color image by using a image recognition technology of convolutional neural network.

14. A wearable device, applicable to a user, comprising:
a main body, comprising a casing, which comprises a sensing surface that can be close to outside skin of the user;
a physiological data sensing module, disposed in the casing, comprising a light emitting unit and a spectrum sensing unit, wherein the light emitting unit is used to emit an incident light of intensity changing with time, the incident light of different intensities enters inside skin of the user to different depths through the sensing surface; the spectrum sensing unit is used to sense an intensity of an outgoing light from inside skin of the user at a series of sampling time to generate a spectrum data set, the spectrum data set comprises a plurality of light intensity data values, the plurality of light intensity data values can be divided into a plurality of groups of depth-light intensity data values respectively obtained corresponding to the plurality of incident light intensities; and
an arithmetic unit, with signal connecting to the physiological data sensing module, for receiving the spectrum data set; according to a signal quality of the plurality of groups of depth-light intensity data in the spectrum data set, the arithmetic unit selects at least one of a first group of depth-light intensity data values satisfying a signal quality index in the spectrum data set to perform a data interpretation at a first judgment time point, and selects at least one of a second group of depth-light intensity data values satisfying the signal quality index in the spectrum data set to perform the data interpretation at a second judgment time point.

15. A method for selecting and interpreting light intensity data values, applicable between a user and a wearable device, the wearable device comprises a light emitting unit and a spectrum sensing unit, the method comprising following steps of:
controlling the light emitting unit simultaneously emit a mixed light comprising multiple spectrum frequency bands, and the mixed light enter inside skin of the user through a sensing surface;
controlling the spectrum sensing unit sense an intensity of an outgoing light from inside skin of the user at a series of sampling time to generate a spectrum data set, wherein the spectrum data set comprises a plurality of light intensity data values, the plurality of light intensity data values can be divided into a plurality of groups of frequency band-light intensity data values respectively obtained corresponding to the plurality of spectrum frequency bands; and
according to a signal quality of the plurality of groups of frequency band-light intensity data in the spectrum data set, selecting at least one of a first group of frequency band-light intensity data values satisfying a signal quality index in the spectrum data set to perform a data interpretation at a first judgment time point, and selecting at least one of a second group of frequency band-light intensity data values satisfying the signal quality index in the spectrum data set to perform the data interpretation at a second judgment time point.

16. A method for selecting and interpreting light intensity data values, applicable between a user and a wearable device, the wearable device comprises a light emitting unit and a spectrum sensing unit, the method comprising following steps of:
controlling the light emitting unit emit an incident light of intensity changing with time, wherein the incident light of different intensities enters inside skin of the user to different depths through the sensing surface;
controlling the spectrum sensing unit sense an intensity of an outgoing light from inside skin of the user at a series of sampling time to generate a spectrum data set, wherein the spectrum data set comprises a plurality of light intensity data values, the plurality of light intensity data values can be divided into a plurality of groups of depth-light intensity data values respectively obtained corresponding to the plurality of incident light intensities; and
according to a signal quality of the plurality of groups of depth-light intensity data in the spectrum data set, selecting at least one of a first group of depth-light intensity data values satisfying a signal quality index in the spectrum data set to perform a data interpretation at a first judgment time point, and selecting at least one of a second group of depth-light intensity data values satisfying the signal quality index in the spectrum data set to perform the data interpretation at a second judgment time point.
